(19) United States
(12) Patent Application Publication
(10) Pub. No.: US 2013/0000647 A1
(43) Pub. Date: Jan. 3, 2013

(54) RESPIRATORY RESISTANCE SYSTEMS AND METHODS

(75) Inventors: Liam Holley, Marrickville (AU); Steven Paul Farrugia, Lugarno (AU); Klaus Henry Schindhelm, Glendaven (AU); Dmitri Anatolievich Doudkine, Chatswood (AU); Paul Andrew Dickens, Springwood (AU); Paul Jan Klasek, Bonnyrigg Heights (AU); Robert Henry Frater, Lindfield (AU)

(73) Assignee: ResMed Limited, Bella Vista (AU)

(21) Appl. No.: 13/497,617

(22) PCT Filed: Sep. 22, 2010
(86) PCT No.: PCT/AU2010/001240

§ 371 Date: Mar. 22, 2012

Related U.S. Application Data
(60) Provisional application No. 61/272,408, filed on Sep. 22, 2009.

(51) Int. Cl. A61M 16/06

(52) U.S. Cl. 128/207.13

(57) ABSTRACT

A patient interface includes a mask configured to communicate with at least one airway of a patient, the mask including at least one aperture (402) configured to deliver gas to the at least one airway of the patient; and an airflow resistance member (400) provided to the mask to control the airflow through the at least one airway.

### CROSS REFERENCE TO PRIORITY APPLICATIONS

This application claims priority to U.S. Provisional Application No. 61/272,408 filed 22 Sep. 2009, which is incorporated herein by reference in its entirety.

### FIELD OF THE INVENTION

The present invention relates to patient interface systems and methods for controlling the flow of breathable gas to a patient. Specifically, the present invention relates to systems and methods for reducing occurrences of snoring and Obstructive Sleep Apnea through the use of controlling the flow of air to a patient.

### BACKGROUND OF THE INVENTION

The loud rumbling, occasionally heard from a sleeping person, may be the result of a particularly loud snoring episode. Snoring is caused by the vibration of the respiratory walls of a person's airway. This vibration then gives rise to the resulting snoring episode. The vibration is caused by obstruction in the movement of air while a person breathes during sleep. The obstruction results from a decrease in pressure between the respiratory walls of the person. Specifically, as the velocity of air passing between the respiratory walls increases, the pressure between the respiratory walls drops. This, in turn, triggers a constriction of the respiratory walls towards each other, which then triggers a snoring episode.

The loud rumbling that occasionally accompanies a snore can be very problematic for people trying to sleep within hearing range of the snorer. However, in addition the effects that snoring has on third parties, snoring may also provide negative consequences to the snoerer. In particular, certain studies have indicated snoring may affect various aspects of a person's quality of life (e.g., through not sleeping well).

To combat the snoring problem, various treatments may be available. Most of the treatments involve clearing the blockage (e.g., the constricted respiratory walls described above) and allowing a person to breathe better while sleeping. Such treatments may include surgery on the collapsing airway (e.g., by the removal of tissue to expand airway), usage of products that control the position of a person's lower jaw or tongue (e.g., a mandibular advancement splint), or pharmaceutical products.

More severe snoring episodes may cause the respiratory walls of a person to completely collapse. Such collapses may lead to and/or be an indication of obstructive sleep apnea (OSA). The resulting collapse of the respiratory walls may then cause misses or pauses in the breathing cycle. The lack of oxygen resulting from a missed breathing cycle may lead to other detrimental consequences for the person. After too many missed breathing cycles, the body may react and cause the person to wake temporarily in order to open the obstructed airway. However, once the person again falls asleep the cycle may again repeat. This ongoing cycle of collapsed airway, missed breathing, sleep disruption may continue throughout the sleep time of the affected person. As a result of this repeating cycle, not only may others suffer from sleep deprivation (e.g., the load rumbling), but the person affected may also suffer from sleep deprivation because of the constant sleep interruptions caused by the collapsed airway.

Various forms of treatment have been developed over the years to address the collapse of the respiratory airways of a patient. One form of conventional treatment for OSA involves the use of positive airway pressure (PAP). Such treatment is disclosed in U.S. Pat. No. 4,944,310. Treatment using PAP, which may be continuous PAP (CPAP), involves the use of a patient interface, which is sealed against the patient's face, to provide a flow of breathable gas and continuous pressure to the respiratory system of a patient. The forced air pressure between the respiratory walls of the patient helps to keep the walls from collapsing.

When a mask is attached to a patient, a flow of breathable gas may be provided from a ventilator machine. This flow of breathable gas provides positive air pressure to force open the respiratory walls of the patient. Thus, conventionally, one approach in addressing snoring or OSA is to externally increase the air pressure of the flow of gas provided to the respiratory area of the patient in order to maintain the pressure between a patient's respiratory walls.

Also known is the “Provent” device by Ventus Medical that fits in the nostril and incorporates a membrane-based microvalve that opens on inspiration and closes on expiration. However, such a device may be uncomfortable from the user's perspective, especially before the user falls asleep.

A patient interface conventionally includes a mask portion. The mask portion may include different types of masks, for example, nasal masks, full-face masks, and nozzles (sometimes referred to as nasal pillows or puffs), nasal prongs, and nasal cannulae, etc.

### SUMMARY OF THE INVENTION

One aspect relates to treatment of snoring, e.g., by reducing the flow of gas inhaled through at least one airway of a patient. Such treatment may be used in conjunction with a mask, although other techniques may not use a mask.

In one form of the present technology a system is provided which controls or limits the peak inspiratory flow.

In one form of the invention a system is provided which prevents or reduces the collapse of the upper airway.

In one form of the invention, a system is provided which breaks a cycle of increasing collapse of the upper airway that may occur with increasing flow velocity.

In one form of the present invention, inhalation resistance is increased, whereas exhalation resistance is left unchanged.

A further aspect relates to controlling the flow velocity of a gas that passes through at least one airway of a patient. An additional aspect may include control of the flow velocity during inhalation by the patient. In addition, in another aspect, the flow velocity of the gas may be controlled during exhalation by the patient.

In certain exemplary embodiments a patient interface is provided. The patient interface may include a mask configured to communicate with at least one airway of a patient. The mask includes at least one aperture to configured to deliver gas to the at least one airway of the patient. The patient interface may further include an airflow resistance member provided to the mask such that breathing by the patient reduces airflow and/or increases impedance during at least inhalation through the at least one airway. The mask may be a nasal mask that defines a substantially sealed breathing cavity over the nasal area of the patient, a full-face mask, or nozzles to interface with the nares of a patient.

Yet another aspect relates to providing the airflow resistance member to control inspiration of the patient, e.g., by placing the airflow resistance member in communication with at least one airway of the patient, such as placing the airflow resistance member on and/or within at least one aperture associated with the mask. The airflow resistance member may be made from a flexible material. However, the airflow resistance member may take the form of a ball shaped object or a porous membrane.

Another aspect relates to disposing a dissolvable structure with the airflow resistance member, such that as the dissolvable structure dissolves the airflow resistance increases.

In one form of the present technology a restriction is provided, the effect of which changes with time. For example, there may be no initial restriction, however the restriction may increase with time.

Yet another aspect relates to configuring the airflow resistance member to structurally respond to a decrease in pressure by further limiting the flow of gas to the at least one airway of the patient.

One form of the present system is adaptive, altering the airflow resistance dependent upon a change in the pressure or the cross-sectional area of the airway.

In other certain exemplary embodiments a patient interface is provided. The patient interface may include a mask configured to communicate with at least one airway of a patient, the mask including at least one aperture to configured to deliver gas to the at least one airway of the patient. The patient interface may include an airflow resistance member provided to the mask configured to be selectively switched between: 1) flow reduction during inhalation by a patient; and 2) flow reduction during exhalation by the patient.

In further exemplary embodiments a method of treatment for snoring is provided. A patient interface is provided to a patient, the patient interface including a mask for communicating with (e.g., fitting over or within) at least one airway of the patient. The flow resistance of gas through the patient interface to the at least one airway of the patient is controlled such that the flow of gas is restricted during at least inspiration of the patient.

According to another example of the present technology, there is provided a patient interface comprising a mask configured to communicate with at least one airway of a patient, the mask including at least one aperture configured to permit entry of gas to the at least one airway of the patient, an airflow resistance member provided to the mask such that, in use, breathing by the patient reduces airflow and/or increases impedance during at least inhalation, and optionally also expiration, through the at least one airway, and progressive airflow resistance structure to cooperate with the airflow resistance member, such that, in use the flow of gas during inspiration and/or expiration is progressively decreased and/or impedance is progressively increased.

In another exemplary embodiment a method for limiting the collapse of a patient's airway between the throat and the soft palette of a patient is provided. A gas flow limiter is provided to the patient such that the gas flow limiter limits the gas flow rate and/or increased impedance to the airway of the patient during inspiration and/or expiration of the patient.

According to another example of the present technology, there is provided a respiratory assistance apparatus for a user, comprising an airflow resistance member to increase impedance and/or limit air flow to the user during inhalation through at least one airway of the user.

According to another example of the present technology, there is provided a respiratory assistance apparatus comprising an airflow resistance member provided to the mask such that, in use, breathing by a patient reduces airflow and/or increases impedance during at least inhalation through the at least one airway, and progressive airflow resistance structure to cooperate with the airflow resistance member, such that, in use the flow of gas during inspiration is progressively decreased and/or impedance is progressively increased.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIGS. 1A and 1B shows illustrative views of an exemplary respiratory system;

FIG. 2 shows an illustrative graph representation of a constant flow of air through an exemplary respiratory system;

FIGS. 3A and 3B show illustrative comparison graphs of measurements from an illustrative respiratory system;

FIGS. 4A, 4B, and 4C show illustrative views of a patient interface with a ball valve according to certain exemplary embodiments;

FIGS. 5A and 5B show illustrative views of a patient interface with an attached leaflet valve according to certain exemplary embodiments;

FIGS. 5C and 5D show illustrative views of a patient interface with an attached porous member or leaflet valve according to certain exemplary embodiments;

FIG. 6 shows an illustrative view of a patient interface device according to certain exemplary embodiments;

FIG. 7 shows an illustrative view of a patient interface device attached to the nasal area of a patient according to certain exemplary embodiments;

FIGS. 8A, 8B and 8C show illustrative views of a progressive patient interface according to certain exemplary embodiments; and

FIGS. 9A and 9B show illustrative views of a variable patient interface according to certain exemplary embodiments.

### DETAILED DESCRIPTION OF ILLUSTRATED EMBODIMENTS

The following description is provided in relation to several embodiments which may share common characteristics and features. It is to be understood that one or more features of any one embodiment may be combinable with one or more features of other embodiments. In addition, any single feature or combination of features in any of the embodiments may constitute an additional embodiment.

The exemplary embodiments described herein may relate to patient interface systems and methods for controlling the flow of breathable gas to a patient. Certain exemplary embodiments may relate to a patient interface in the form of a nasal resistor to restrict the flow to gas through (to and/or from) a patient's respiratory walls. Certain exemplary techniques may include methods of treatment for snoring and/or OSA through the use of restricting airflow to the respiratory walls of a patient. In other exemplary embodiments, the flow restriction can be attached or otherwise provided to existing masks (e.g., retrofit).

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The term “air” will be taken to include breathable gases, for example air with supplemental oxygen. It is also acknowledged that the blowers described herein may be designed to pump fluids other than air.

### Overview

As stated earlier, one cause of snoring and OSA may be linked to the constriction and/or collapse of a patient's respiratory walls. This collapse may be partially explained by an application of Bernoulli's Effect on the system of the patient's respiratory passage. Specifically, as the velocity of air increases between the respiratory walls of the patient a corresponding drop in pressure between the respiratory walls may occur.

In some patients, as the result of increasing flow velocity, there can be a reduced air pressure in the upper airway, which in turn can lead to a reduction in cross-section of the upper airway, this in turn increases the velocity for a given volumetric flow rate, in turn decreasing the pressure and further reducing the cross-section of the airway, which eventually may lead to the complete collapse of the airway. Thus there can be a cycle of positive feedback, giving rise to further restrictions. A device in accordance with the present technology can break this cycle of positive feedback by controlling or limiting the flow.

In accordance with the present technology, patients can acclimatize with the presence of a restriction at the entrance to the airway and may prevent the onset the positive feedback cycle described above.

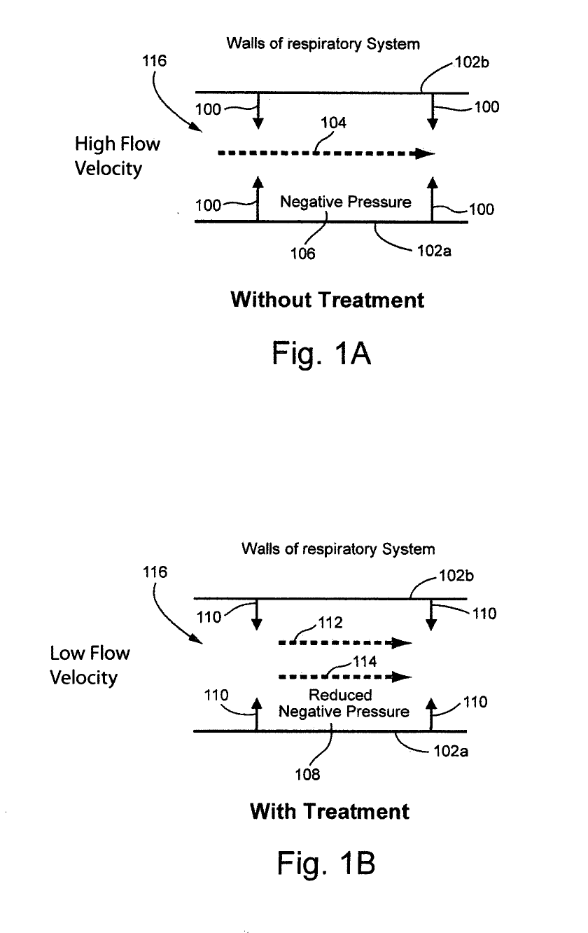

Referring now to FIG. 1A, an illustrative view with exemplary walls of an exemplary respiratory system is shown. A flow of air is shown entering intake point 116. Intake point 116 may be subject to high flow velocity, for example due to low intake impedance. Respiratory walls 102a and 102b are shown with a flow of air moving at a high velocity, represented by arrow 104, moving between respiratory walls 102a and 102b. The flow of air moving at a high velocity through the respiratory system may be caused by a relatively large volume of air trying to flow through the respiratory system. As explained above, the Bernoulli Effect correlates a high velocity flow of air to low pressure areas. The high velocity flow of air through respiratory walls results in negative pressure 106, which may be a pressure lower than normal atmospheric pressure. As shown by arrows 100, in response to negative pressure 106, respiratory walls 102a and 102b may constrict, especially when in a relaxed state. The resulting constriction of respiratory walls 102 and 102b may then lead to a snoring episode or further occurrences of OSA.

It is believed that this constriction effect is more pronounced on inhalation than on exhalation, as during inhalation the airways are at lower than atmospheric pressure to create a negative pressure gradient for air to flow into the lungs, whereas during exhalation the airways are at greater than atmospheric pressure to create a positive pressure gradient. The embodiments of the present invention therefore relate primarily to means for modifying flow velocity into/through the airways on inhalation, although the means may also operate to modify flow velocity on exhalation.

Preferably, the flow velocity modifying means acts differentially during inhalation and exhalation, so as to preferentially restrict air flow velocity during inhalation compared to during exhalation.

FIG. 1B shows an illustrative view with exemplary walls of an exemplary respiratory system. In contrast to the above illustrative view in FIG. 1A, FIG. 1B may have lower flow velocity at intake point 116, for example due to higher intake impedance. This higher intake impedence may result in a lower flow velocity passing through the respiratory system. Accordingly, the velocity flow of air, represented by arrows 112 and 114, moving between respiratory walls 102a and 102b may be lower than the velocity shown in FIG. 1A. This lower velocity flow may in turn result in reduced negative pressure 108 and less constriction, represented by arrows 110, between respiratory walls 102a and 102b. As can be seen in FIG. 1B, arrows 112 and 114 represent lower velocity flow. However, the lower velocity slow is offset by the increased area available for the air to pass through the respiratory system (as can be seen by the two arrows in FIG. 1B vs. the one arrow in FIG. 1A). Accordingly, the total flow volume or air passing through the exemplary respiratory system may be the same or higher than shown in FIG. 1A.

Further, snoring episodes and OSA occurrences may be countered because respiratory walls 102a and 102b in FIG. 1B are not as constricted. It will be appreciated that there are various techniques that may be implemented that reduce the velocity of air flow through a respiratory system. In the above illustrative view increased impedence at the intake point of the respiratory system results in a lower velocity flow through the respiratory system. However, alternative techniques may also be applied. Such techniques may include, for example, attaching a blower to the intake point (e.g., through a tube) and using the blower to control a reduction in the velocity of airflow through the respiratory system.

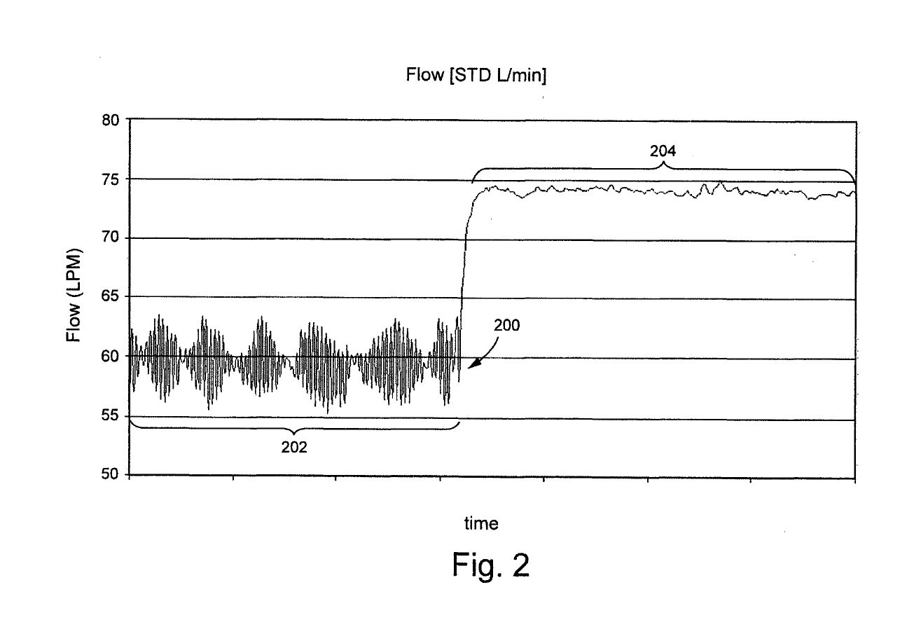

FIG. 2 shows an illustrative graph of a constant flow of air through an exemplary respiratory system. The illustrative graph of FIG. 2 may be accomplished by using a starling resistor coupled with a flow generator and a flow computer (e.g., a patient's respiratory system is physically simulated with a starling resistor and then measured with software). In this illustrative graph representation an exemplary respiratory system is provided with an initial constant differential pressure across the system, and the rate of flow through the system is measured (as shown in FIG. 2). Initially, as seen in section 202, there is an erratic throughput of flow through the exemplary respiratory system. This can be considered a simulated snore. At point 200 the intake area of the exemplary respiratory system was partially occluded while the pressure differential across the system was kept constant. It will be appreciated that other techniques of increasing intake impedance may be utilized (e.g., reducing the pressure from a flow generator). Once the intake impedance is increased the overall throughput of the exemplary respiratory system may jump. In the illustrative graph representation, this is seen by comparing section 202, which averaged around 60 LPM, to section 204, which averaged around 75 LPM. Thus, an increase in impedance at the intake flow point may result in an overall lower impedance rate for a total exemplary respiratory system.

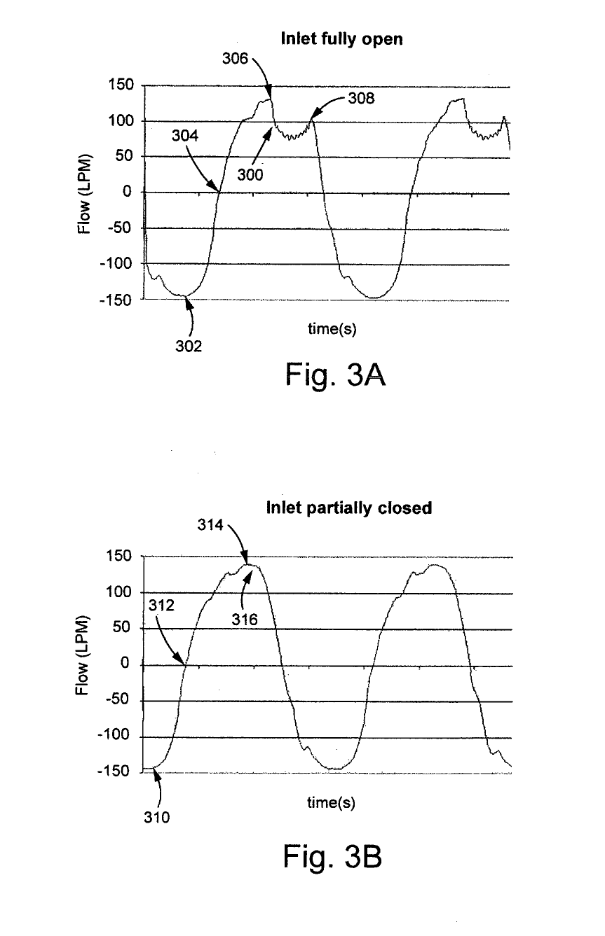

FIGS. 3A and 3B show comparison graphs of measurements from an exemplary respiratory system (e.g., a patient's respiratory system is physically simulated and then measured with software). FIG. 3A shows an illustrative respiratory pattern where the flow inlet to the exemplary respiratory system is fully open. At point 302, the exemplary respiratory system is in the middle of the expiratory phase. At point 304, the expiratory phase of the exemplary respiratory system transitions to the inspiration phase of the exemplary respiratory system. From point 304 to point 306 the inspiration flow rate increases in the exemplary respiratory system. At point 300, however, at or about the peak of inspiration, a snoring episode in the exemplary respiratory system occurs, resulting in a drop in the overall flow rate of the respiratory system. The exemplary respiratory system recovers at point 308 and then continues in its transition back to the expiratory phase, eventually repeating the same “snoring episode” again, later in time.

In contrast to FIG. 3A, FIG. 3B shows an illustrative respiratory pattern where the flow inlet to the exemplary respiratory system is partially closed. At point 310 the expiratory phase is at a maximum flow rate and begins to decline to point 312 where the transition between expiration and inspiration in the exemplary respiratory system occurs. The inspiration rate gradually climbs to point 314. However, unlike the illustrative respiratory pattern shown in FIG. 3A, at point 316, no snoring episode occurs at the peak of inspiration. Thus, partially closing or restricting the airflow inlet valve for the exemplary respiratory system may prevent snoring episodes. In other words, increased impedence at the intake point may result in lower velocity air flow through the respiratory system. However, the lower velocity air flow may (as seen in FIGS. 2 and 3B) result in an overall increase in flow volume through the respiratory system due to increased air passage diameter.

The above illustrative techniques may be carried out in one or more exemplary embodiments. Certain exemplary embodiments utilizing the above illustrative techniques are described below.

### Ball Valve Embodiment

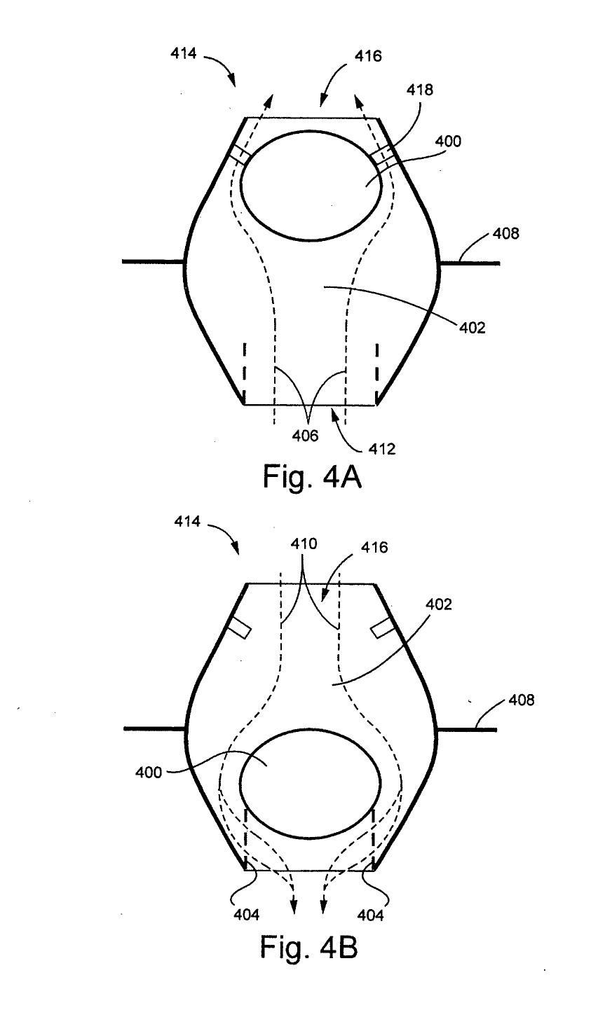

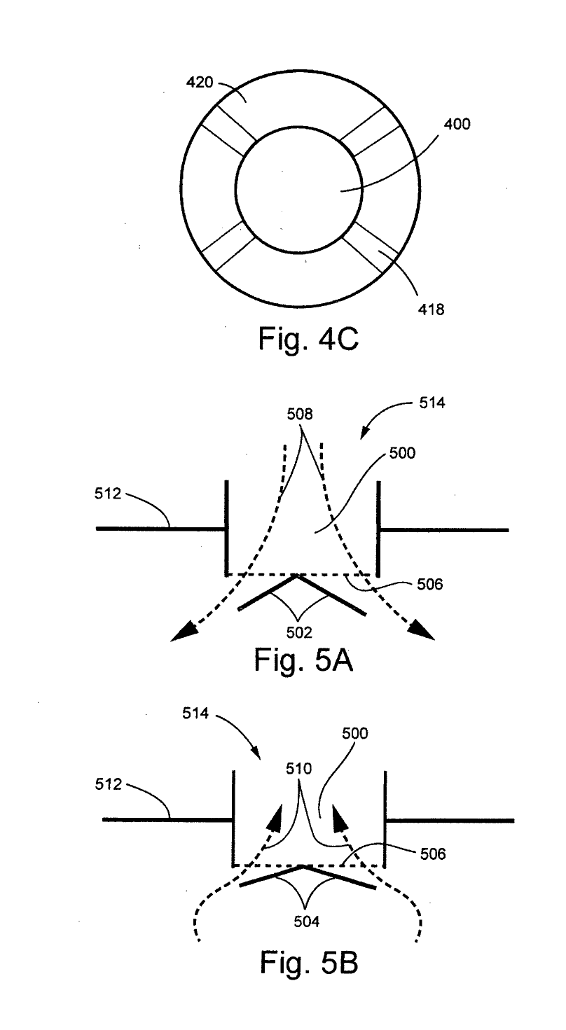

FIGS. 4A and 4B show illustrative views of a patient interface with a ball valve according to an exemplary embodiment. Patient interface 414 defines a structure to form a substantially oval air cavity 402. Patient Interface 414 may be configured to cooperate with the nasal area of a patient. It will be appreciated that various techniques may be used such that patient interface 414 may fit over or otherwise engage with the patient. For example, the patient interface 414 may fit the inside of and/or in the vicinity of the nostril, over the mouth area, over the mouth only, over the mouth and nose area, etc.

As shown in FIGS. 4A and 4B, ball 400 is disposed within air cavity 402. Other object shapes may be utilized instead of, or in addition to, ball 400. Such objects may include, for example, oval shaped objects, cubed shaped objects, etc. In the case where patient interface 414 is in the form of a nozzle, cannula or prong, one end of the interface 414 can be inserted at least a small amount into the patient's nares, in which case ball 400 may be disposed to move at least partly within the nasal cavity of a patient. Each nare nozzle can be independent, or a pair of nozzles can be formed to a common plenum, which in turn includes at least one aperture for supply of gas, ambient or otherwise.

As shown in FIG. 4A, ball 400 is provided to reduce airflow 406 provided via inlet 412 during inspiration by the patient. This is accomplished by ball 400 partially occluding outlet 416. Outlet 416 may interact with the nose of the patient. Ball 400 may respond to airflow 406 during inspiration and may move up air cavity 402 toward outlet 416 which may communicate with an air passage of the patient (e.g., a nare of the patient). One or more supports 418 may be provided to prevent ball 400 from completely blocking airflow 406 through outlet 416. Thus, as ball 400 comes into contact with prongs 418, airflow 406 becomes partially restricted. It will be appreciated that prevention of complete occlusion of airflow 406 at outlet 416 may be accomplished by utilizing other techniques. Such techniques may include, for example, providing ball 400 with a shape that differs from shape of outlet 416, to ensure airflow 406 is not be completely blocked, providing prongs on ball 400, providing a ball with grooves that facilitate the passage of airflow 406 through outlet 416, etc.

As shown in FIG. 4B, certain exemplary embodiments may provide for substantially unimpeded airflow during patient expiration. In FIG. 4B, patient interface 414 is shown during expiration by the patient. Airflow 410 illustratively shows the expiration pathway taken through air cavity 402, around ball 400, and through expiration vents 404. It will be appreciated that expiration vents 404 may be provided as one-way expiration vents only allowing air flow out during expiration but not during inspiration. As seen in the illustrative view of FIG. 4B, ball 400, reacting to the expiratory airflow and/or gravity (e.g., gravity may provide the location of ball 400 with a “default” position within the air cavity), is moved down and away from air passage 416 and down to inlet 412. While the path taken by airflow 406 in FIG. 4A may be substantially closed off by ball 400, expiration vents 404 are substantially unimpeded, and are dimensioned to have an overall cross-sectional area that allows the substantially unimpeded expiration of air, as shown by airflow 410, by the patient.

Patient interface 414 may be attached to a patient through the use of adhesive seal 408. Such adhesive seals may be disclosed in commonly owned U.S. patent application Ser. No. 12/478,537 filed Jun. 4, 2009, the contents of which are herein incorporated by reference. Adhesive seal 408 attaches to the skin of a patient and may in-turn facilitate the attachment of patient interface 414 to adhesive seal 408. Thus, patient interface 414 may be held to the nasal and/or face area of a patient, e.g., the rim of the nostril.

FIG. 4C shows a perspective view of a patient interface device utilizing a ball according to certain exemplary embodiments (e.g., looking down on ball 400 in FIG. 4A). As explained above, ball 400 may engage supports 418 to prevent ball 400 from completely occluding the passage of airflow to the airways of a patient during inspiration. Gaps 420 are formed by prongs 418 in conjunction with ball 400 and allow for restricted airflow 406 to pass between prongs 418 and into the airways of a patient.

### Leaflet Valve Embodiment

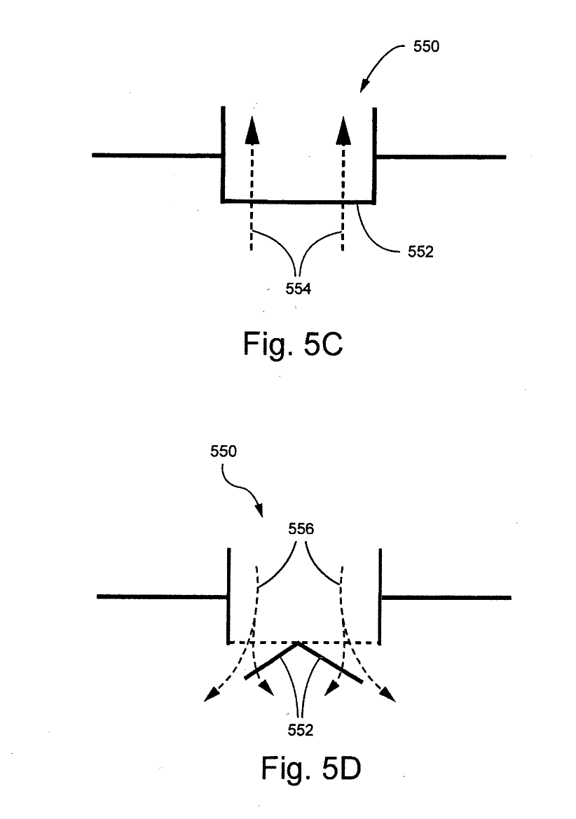

Referring now to FIGS. 5A and 5B, illustrative views of a patient interface with an attached leaflet valve according to certain exemplary embodiments are shown. Patient interface 514 defines a structure containing air cavity 500. FIG. 5A shows an illustrative view of an exemplary patient interface during expiration by a patient. Arrows 508 show the illustrative airflow during expiration by the patient. During expiration a valve, e.g., a leaflet valve 502 (having one or more flaps), responds (e.g., bends, pivots and/or flexes) to the expiratory airflow and/or gravity by opening such that the expiratory airflow from the patient is substantially unimpeded. In contrast, as shown in the illustrative view of FIG. 5B, during inspiration arrows leaflet valve 502 responds by biasing towards air cavity 500. The results of the biasing may lead to a decrease in the amount of flow, as shown by air flow lines 510, so as to reduce air flow velocity in the downstream respiratory passageways during inhalation. Thus, leaflet valve(s) “closes” and restricts the overall intake of airflow by the patient during inspiration. Alternatively, or in addition, dedicated airflow vents (not shown) that may not be covered and/or impeded by leaflet valve 502 may be provided. Such vents may facilitate the prevention of complete inspiration or expiration resistance.

It will be appreciated that leaflet valve 502 may interact with structures that define other types of air cavities. For example, nozzles may be configured to interact with the nares of a patient. One or more leaflet valves may then be positioned within each of the nozzles, e.g., at either end of the nozzles, or a single valve may be provided for both nozzles collectively in order to restrict the flow of air during inspiration and/or expiration by the patient and thus subsequently lower the velocity of the flow of air through the patient's respiratory system.

At the entrance to air cavity 500 supporting structure 506 is provided. Supporting structure 506 is attached to the general structure of patient interface 514. Leaflet valve 502 is connected to and supported by supporting structure 506. Leaflet valve is further structured such that the flaps thereof partially close over the entrance to air cavity 500 during inhalation by the patient. This results in a reduced flow of air through the air cavity and subsequently into the patient. During exhalation the flaps/valve may be substantially open, providing substantially unimpeded exhalation airflow. At normal air pressure (e.g., no air flow) leaflet valve/flaps may be in a default position as shown in FIG. 5A. It will be appreciated that the “default” position of the leaflets may be modified to suit certain embodiments. For example, the position of leaflets in FIG. 5B may be default position, other positions may also be the default position (e.g., in between the position of leaflet valve as shown in FIGS. 5A and 5B).

Patient interface 514 may be connected to the patient through the use of seal 512, which may include adhesive. Supplemental or alternative techniques may include, for example, structuring the walls of patient interface 514 to fit within a nare of the patient and sealingly engage with the nostril. It will be appreciated that other techniques (e.g., strap systems) may also be utilized for holding patient interface 514 to the face of a patient.

The configuration of the leaflet valves may be altered from the exemplary embodiments discussed herein. Such configurations may include, for example, attachment to the patient interface or supporting structure at one end of the aperture (e.g., at the outer portion of the aperture rather than the middle), attachment around the edge of the aperture forming a funnel like restriction for the flow of air (e.g., connecting in a circular pattern around the edge of the air cavity entrance and converging towards a central point), etc. The shape of the leaflet may also be modifiable. Such shapes may include, for example, rectangular, oval, triangular, irregular, etc.

Referring now to FIGS. 5C and 5D, illustrative views of a patient interface with an attached porous valve or member according to certain exemplary embodiments are shown. Patient interface 550 is provided with porous leaflet valve or member 552.

As shown in FIG. 5C, porous leaflet valve 552 is in a closed position. In this exemplary embodiment this position is also the default position. As shown, the porous nature of porous leaflet valve 552 facilitates airflow 554 through patient interface 550 and to the airways of a patient (not shown). The porosity porous leaflet valve 552 may reduce the overall inspiration of air to the airways of a patient between 1 and 50 percent, e.g., 5-20%. Certain exemplary embodiments may utilize materials for the leaflet valve that have a porosity that reduces the overall airflow by around 5 percent during inspiration. Such materials may include, for example, Gore-Tex, various paper materials, polymeric materials, molded silicone, etc.

In contrast to FIG. 5C, FIG. 5D shows porous leaflet valve 552 in a relatively open position, allowing substantially unimpeded expiration of airflow 556. Porous leaflet valve 552 responds to expiratory airflow 556 and opens. The porous nature of porous leaflet valve 552 allows for some of airflow 556 to pass through leaflet valve. Alternatively, or in addition, airflow 556 may pass through the newly opened space created by the opening of porous leaflet valve 552.

The design, material, shape, and configuration of valves 502 and/or 552 may be modified to suit the needs of the patient and/or adjust the flow rate allowed during inhalation or exhalation. Such adjustments may allow a patient to vary the flow rate based on the type or shape of material that is being utilized as the leaflet valve. The material used for leaflet valves may include, for example, porous or non-porous materials, stiff or flexible materials. Such materials may include, for example, paper, Gore-Tex, silicone flaps or membranes, polymeric materials, etc.

### Mask with Leaflet Valve Embodiment

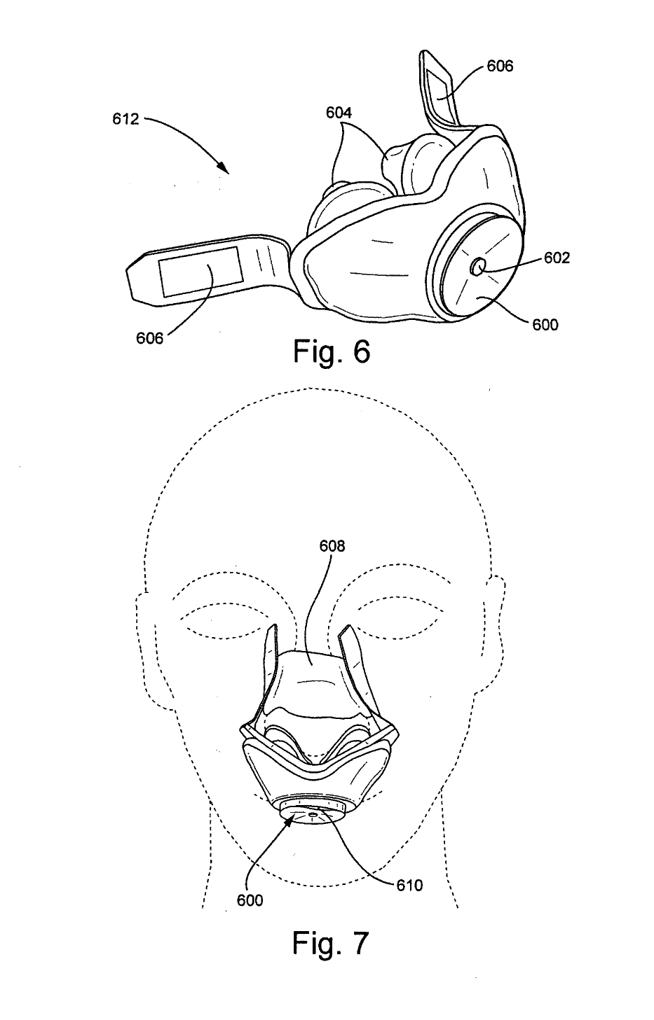

Referring now to FIG. 6, an illustrative embodiment of an exemplary patient interface device is shown. Such an exemplary patient interface device may be disclosed in International Application PCT/AU2008/001557, filed Oct. 22, 2008, the contents of which are herein incorporated by reference. Interface device 612 may include two nasal prongs or nozzles 604, each of which may be configured to interface with a nare of a patient. Nozzles 604 may be configured to form one airflow aperture (not shown). Provided at airflow aperture 610 is airflow resistance valve 600. Airflow resistance valve 600 substantially covers the airflow aperture during inspiration of the patient, thus restricting the airflow to the airways of the patient. Airflow resistance valve 600 may be held in place at the airflow entrance by suitable structure 602 (e.g., a screw or spigot) that may be connected to a beam or cross element that may provided across airflow aperture 610. In this embodiment, the structure 602 is adapted to hold and secure the valve 600 by way of a spigot mount extending through valve 600. Preferably, the valve 600 is a flexible member or leaflet which is able to be deflected by the airway generated either through inspiration and/or expiration. The leaflet during inspiration partially seals the aperture during inspiration, and thereby limits the inflow of air. However during expiration, the leaflet deflects away from the aperture and opens the valve 600, thereby allowing air to freely be exhaled. It will be appreciated that the default “resting” position of the airflow resistance member may be established where the airflow resistance member is closed, where the airflow resistance member is open, or at other positions.

FIG. 7 shows an illustrative view of an exemplary patient interface device attached to the nasal area of a patient. Nozzles 604 interface with the nares of a patient, sealingly forming around the nares of the patient. The irregular shape, structure, and/or placement of the airflow resistance device may form restricted airflow aperture 610. Restricted airflow aperture 610 may allow for restricted airflow during inspiration. In contrast, airflow resistance valve 600 may open to allow substantially unimpeded expiration airflow from the patient. The airflow resistance valve may be formed out of any suitable material. Such materials may include, for example, a piece of paper towel, fabric, a porous membrane, rubber/silicone, etc.

Adhesive seal 608 is provided across the bridge of the patient's nose. Such adhesive seals may be disclosed in commonly owned U.S. patent application Ser. No. 12/478,537 filed Jun. 4, 2009, the contents of which are herein incorporated by reference. The outer layer of adhesive seal 608 is configured to attach to straps 606 to hold patient interface 612 in place. Attachment of straps 606 and outer layer of adhesive seal 608 may be Velcro. However, other techniques for attaching patient interface to the patient may be utilized, for example, a strap system.

It will be appreciated that other combinations may be applied to the above illustrative embodiment. Such combination may include, for example, the patient interface having one or more airflow resistance valves inside each nozzle of the patient interface device, having two nozzles with each having a separate air pathway and providing airflow resistance nozzles at the end of each air pathway, an air cavity may be used instead of two nozzles, etc.

### Progressive Resistance Embodiment

When utilizing a nasal resistor it may be extremely uncomfortable for a patient to breathe when the airways of the patient are affected by the nasal resistor. The increased resistance provided by the nasal resistor to the breathing process of the patient may additionally lead to high rejection rates during treatment or therapy of the patient. Thus, patients seeking to address snoring or OSA may be left untreated.

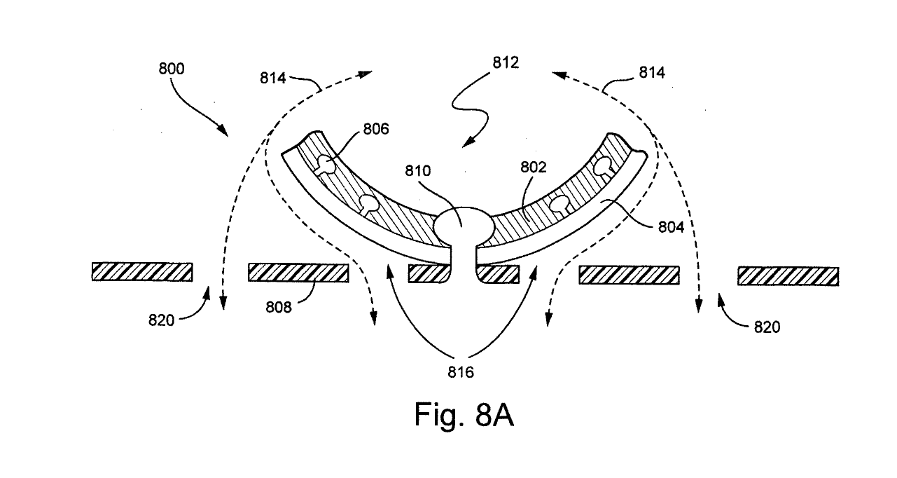

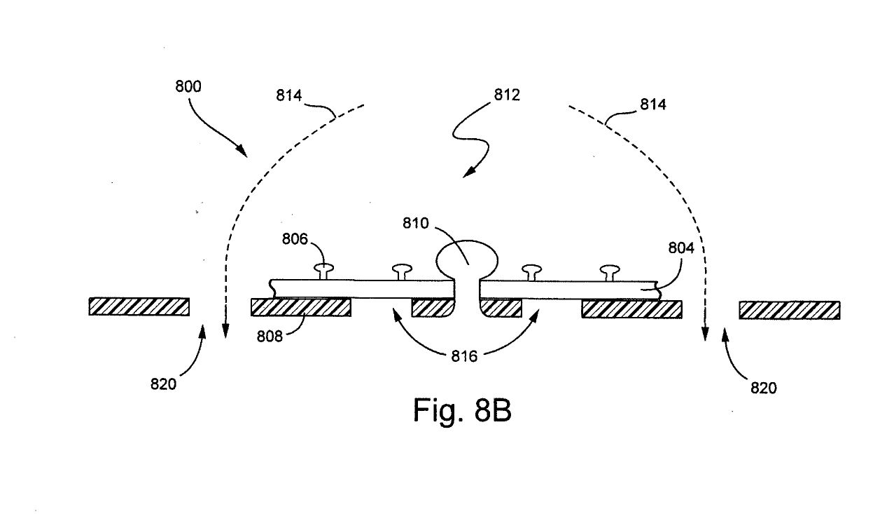

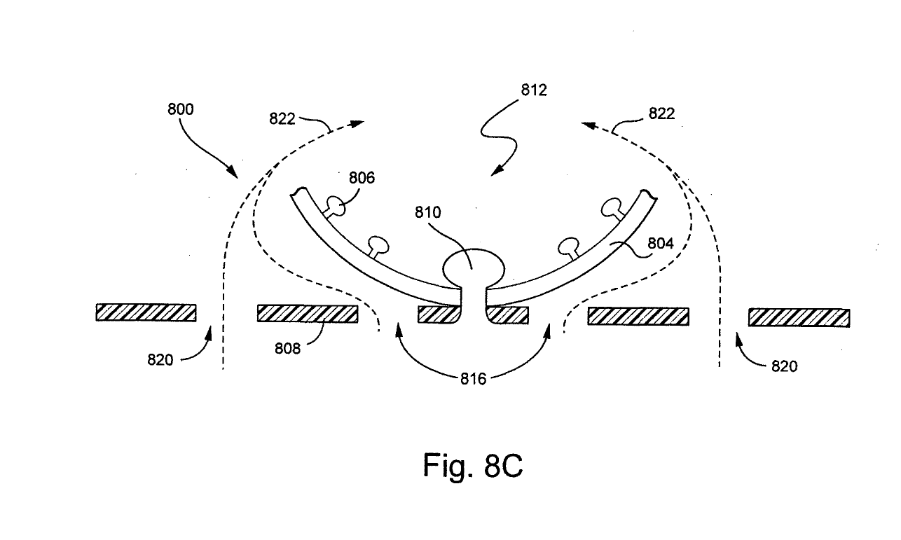

Certain exemplary embodiments may utilize a progressive nasal resistor. Functionally, these certain exemplary embodiments may operate by slowly increasing the resistance of a patient's breathing over a period of time. For example, a patient may put on a nasal resistor such as the one in the exemplary embodiment of FIG. 8A-8C. Initially, while the patient is awake, the resistance to breathing provided by the nasal resistor may be small, facilitating easier breathing by the patient. However, as the patient falls asleep, the breathing resistance may slowly increase using progressive resistance structure or techniques. This increased air flow resistance, as explained above, may then help address snoring episodes or OSA.

FIGS. 8A and 8B show illustrative views of a progressive nasal resistor according to and exemplary embodiment. Nasal resistor 800 may be configured to interface with a nare of a patient. Nasal passage 812 may be partially sealed by nasal resistor 800.

A structure may be constructed to progressively provide resistance to inhalation airflow by the patient. For example, a temporary shape holding member, e.g., a water-soluble polymer 802, may be configured to communicate with the nasal passage 812. The composition of water-soluble polymer 802 may include materials such as, for example, starch, e.g., corn starch, or water soluble plastic. One suitable material is a water-soluble plastic made from corn starch (see www.plantic.com.au—Plantic Technology). Both single use and multiple use compositions are possible. Water-soluble polymer 802 may be semi rigid and may be configured to hold in place flexible material 804. That is to say, flexible material 804 may be forced into a position by the predefined shape of water-soluble polymer 802. Further, keys 806 may be provided on flexible material 804 to add to the adhesion and/or coupling between water-soluble polymer 802 and flexible material 804, e.g., by increasing surface area contact and mechanical locking between the flexible material 804 and polymer 802. It will be appreciated that other techniques may be provided to aid in the adhesion instead of flexible material 804 and water-soluble polymer 802. Such techniques may include, for example, indentations in flexible material 804, increasing roughness on the inner surface of flexible membrane 804, etc. Flexible material 804 may be constructed out of a soft flexible material, such as silicone, a soft plastic, rubber or other flexible material.

A supporting structure, e.g., rigid plastic 808, is provided across the nasal area of a patient. Airway gaps 816 and 820 are formed in rigid plastic frame 808. In FIG. 8A airflow 814 may pass to and from nasal passage 812 through airway gaps 816 and 820. It will be appreciated that these gaps may be small holes provided to allow restricted inspiration, or may be constructed as other types of gaps to facilitate the passage of air between the outside air and the nasal area of a patient. Support structure 810 is provided which attaches to rigid plastic 808, flexible membrane 804, and porous material 802.

As shown in the illustrative view of FIG. 8A, water-soluble polymer 802 may form a substantially concave shape able to communicate with a nare of a patient. The substantially concave shape of water-soluble polymer 802 forces flexible material 804 into a similar concave shape. When held in such a concave shape, the resistance to breathing and the flow of air provided to a patient through the nasal resistor is substantially unimpeded during both expiration and inspiration. Water soluble polymer 802 may be in communication with nasal passage 812. As time passes, e.g., 5-10 minutes or up to one hour or more, water soluble polymer 802 slowly dissolves as it interacts with the humid air of nasal passage 812. The amount of time water soluble polymer 802 dissolves to the point as shown in FIG. 8B may be configured to fit the needs of individual patients. For example, one patient may be provided with a 30 minute ramp time through the dissolvable polymer, while another may be provided with a 1 hour ramp time. As shown in FIG. 8B, the gradual dissolution of water soluble polymer 802 facilitates the gradual straightening of flexible membrane 804. As flexible material 804 becomes less and less concave the resistance to airflow during inspiration slowly increases as gaps 816 are blocked during expiration.

As shown in FIG. 8B, when water soluble polymer 802 substantially or completely dissolves, airway gaps 816 may be completely blocked during expiration. With airway gaps 816 blocked expiratory airflow 814 only proceeds through airway gaps 820. It will be appreciated that the number of gaps provided may be altered to suit the needs of the patient. For example, 20-100 or more air holes (instead of the 4 shown) may be provided and flexible member may cover a certain amount which may decrease the overall expiratory airflow by 1-50% or more, e.g., 1-5% or more, 5-15% or more, 10-30% or more, etc. Other embodiments may adjust the expiratory airflow between 1 and 50 percent. Alternatively, or in addition, rigid plastic may instead be constructed out of a porous material that facilitates the transfer of airflow through 808. Thus, flexible material 804 may only block a portion of the surface area of the porous material and still allow the transfer of air.

FIG. 8C shows an illustrative view during inspiration according to certain exemplary embodiments. Patient interface 800 is shown during inspiration with water soluble polymer completely dissolved. Airflow 822 illustrates the path that the inspiratory airflow may take when patient interface 800 is in such a state. Flexible material 804, reacting to the inspiratory airflow and the resulting pressure change, bends inwards, uncovering air gaps 816. Airflow 822 may then pass through air gaps 816 and 820, facilitating substantially unimpeded airflow 822 during inspiration by the patient.

Thus, a patient may utilize a nasal resistor while awake in relative comfort, and when the patient falls asleep the air flow resistance level may be increased such that snoring or OSA is addressed.

It will be appreciated that while water-soluble polymer is dissolving the relative freedom of movement flexible material 804 is restricted. Thus, when water soluble polymer is partially dissolved the relative airflow resistance may be greater than that provided in FIG. 8A, but less than that provided in FIG. 8B. Such an arrangement can also be used to restrict air flow during inspiration, by rearrangement of the parts such that during expiration all holes are opened, and during inspiration only a subset of those holes are opened.

It will also be appreciated that other configurations of the above embodiment may be implemented. Such configurations may include, for example, gradually increasing inspiratory resistance (e.g., flipping the direction of the flexible material and the water-soluble polymer), increasing expiration and inspiration resistance, etc. Additionally, or alternatively, while the above exemplary embodiment is shown as a single use device other nasal resistors may utilize techniques which allow a person to “reset” the resistance of the nasal resistor after one use. Such multi-use nasal progressive nasal resistors may utilize, for example, a gradual spring to control the level of airflow resistance the flexible membrane provides, a timed gear assembly may also be provided to automatically or manually adjust the level of airflow resistance for the patient.

### Variable Resistance Embodiment

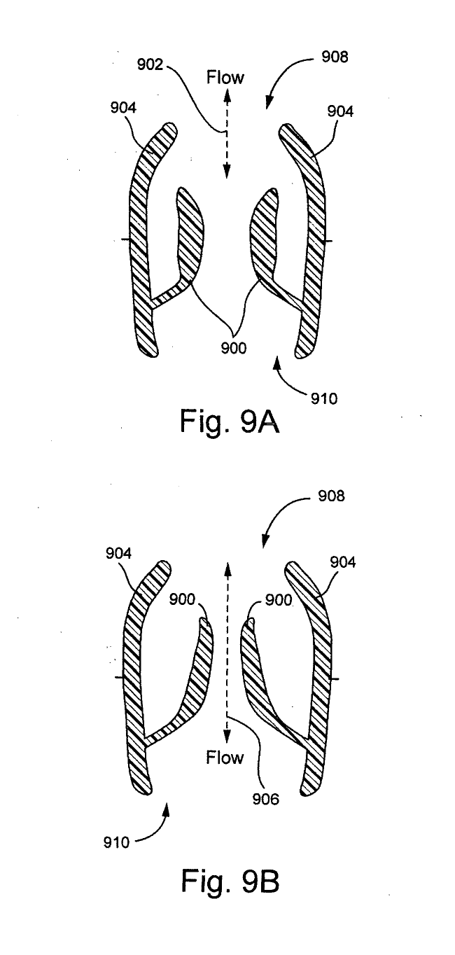

FIGS. 9A and 9B show illustrative views of a variable flow resistance device according to an exemplary embodiment. Structure 904 defines an outer shell to communicate with the walls of a breathing passage, and an outlet 908 and an inlet 910 through which a flow of air may pass. Materials used in forming structure 904 may include, for example, silicone rubber. Outlet 908 may communicate with the airway of a patient and inlet 910 may communicate with a supply of air (ambient) for the patient. The breathing passage may be located within the body of a patient (e.g., a nare), or may be located in a patient interface device (e.g., a nozzle). A pair of variable air flow resistance members 900 may be provided with structure 904. Variable air flow resistance members 900 may be configured such that low pressure between the variable air flow resistance members results in a constriction and overall reduction in airflow rate. The physics of this process may operate similar to the above described exemplary respiratory systems. As shown in FIG. 9A, variable air flow resistance members 900 are relaxed and provide for relatively unimpeded airflow 902. In contrast, FIG. 9B shows an increased velocity in air flow 906 between air flow resistance members 900. This increased velocity may result in a pressure drop between air flow resistance members 900 and a subsequent constriction, as shown in FIG. 9B. The resulting constriction may then decrease the overall airflow through inlet 908 or outlet 910 (e.g., depending on the direction of the air flow 902 or 906).

It will be appreciated that other techniques for adjusting variable resistance in certain exemplary embodiments may be utilized. Such techniques may allow patients to manually adjust the degree of airflow resistance through a dial, switch, or other similar device. It will also be appreciated that the variable flow resistance device may be configured such that air flow resistance members may only impede a particular direction of airflow. Thus, during inspiration air flow may be restricted if there is a high flow of air, but during expiration air flow may be relatively unobstructed.

Preferably, airflow during inspiration is limited or impeded to a greater level than expiration, although the impedance during exhalation can get to be greater than the inhalation impedance. It is also possible to alternate whether the impedance during inhalation or exhalation is higher, and/or it is possible to increase impedance during both inhalation and exhalation.

### Additional Embodiments

Other exemplary embodiments may also be provided. For example, certain exemplary embodiments may utilize a selective switch so as to adjust whether increased inspiration or increased expiration resistance may be used to address the snoring episodes or an OSA condition of a patient. Thus, a patient and/or physician may try out each setting (reduced inspiration or reduced expiration) to find a setting that may work for a given patient.

Certain exemplary embodiments may provide mouthpiece patient interfaces. Such interfaces may include grooves in which a patient's teeth and or gums are positioned to hold the interface in place. The interface may be provided with small holes to facilitate breathing by a patient. Such interfaces alternatively, or in addition, may control the rate of airflow to the respiratory system through mouth of the patient in a manner similar to the above described embodiments. Mouthpiece patient interfaces may also facilitate increased flow resistance in the mouth of a patient relative to that provided by an exemplary nasal resistor.

Further exemplary embodiments may use a patient interface device attached to a blower to control the velocity of airflow through a patient's respiratory system.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment. In addition, while the invention has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

1. A patient interface comprising:
a mask configured to communicate with at least one airway of a patient, the mask including at least one aperture configured to deliver gas to the at least one airway of the patient; and
an airflow member provided to the mask configured to reduce a flow velocity of the delivered gas through the at least one airway to reduce or eliminate snoring.

2. The apparatus of claim 1, wherein the mask is a nasal mask and defines a substantially sealed breathing cavity over the nasal area of the patient.

3. The apparatus of claim 1, wherein the mask includes at least one exhalation vent.

4. The apparatus of claim 1, wherein the at least one airway is at least one nare of the patient.

5. The apparatus of claim 4, wherein the mask comprises at least one nozzle to interface with the at least one nare of the patient.

6. The apparatus of claim 5, wherein the at least one nozzle comprises a pair of nozzles to interface with the respective nares of the patient.

7. The apparatus of claim 6, wherein the at least one aperture of the mask is one aperture and the pair of nozzles merge to communicate with the one aperture of the mask.

8. The apparatus of claim 6, wherein the at least one aperture is a pair of apertures and each one of the pair of nozzles forms communicates with the pair of apertures, respectively.

9. The apparatus of claim 2, wherein the airflow member is provided within the substantially sealed breathing cavity formed by the mask.

10. The apparatus of claim 3, wherein the airflow member is configured to provide substantially unimpeded airflow through the at least one exhalation vent.

11. The apparatus of claim 1, wherein the airflow member is provided on and/or within the at least one aperture.

12. The apparatus of claim 5, wherein the airflow member is provided within the at least one nozzle.

13. The apparatus of claim 1, wherein the airflow member is composed of a flexible material.

14. The apparatus of claim 1, wherein the airflow member is a ball shaped object.

15. The apparatus of claim 1, wherein the airflow member is a porous membrane.

16. The apparatus of claim 13, wherein the flexible material is configured such that during inspiration by the patient the flexible material is more closed and allows less gas through the at least one aperture relative to expiration by the patient.

17. The apparatus of claim 16, wherein the at least one aperture further comprises a supporting structure to support the flexible material provided to the mask.

18. The apparatus of claim 15, wherein the porous membrane is formed of a polymer based material.

19. The apparatus of claim 1, wherein the airflow member includes a progressive airflow resistance structure that is structured to cooperate with the airflow member, such that, in use, airflow during inhalation progressively decreases.

20. The apparatus of claim 19, wherein the progressive airflow resistance structure includes dissolvable structure including a water soluble polymer that dissolves over time, thus allowing the airflow member to reduce airflow to the patient in use.

21. The apparatus of claim 20, wherein the water soluble polymer comprises corn starch.

22. The apparatus of claim 20, wherein the airflow member comprises indentations to increase the bonding strength between the dissolvable structure and the airflow member.

23. The apparatus of claim 1, wherein the airflow member structurally responds to a decrease in pressure by further limiting the supply of gas supplied to the at least one airway of the patient.

24. The apparatus of claim 23, wherein the air flow resistance member comprises at least one prong that structurally responds to the decrease in pressure.

25. The apparatus of claim 1, wherein the mask attaches to the nasal area of a patient with the use of an adhesive seal.

26. The apparatus of claim 1, wherein the mask attaches to the patient with a plurality of interlocking straps.

27. The apparatus of claim 1, wherein the airflow member further provides substantially unimpeded airflow during exhalation.

28. The apparatus of claim 1, wherein the reduction in airflow is selectively controllable.

29. The apparatus of claim 1, wherein airflow during inhalation further decreases over a period of time.

30. The apparatus of claim 29, wherein the patient can reset the inhalation resistance provided by the airflow member.

31. The apparatus of claim 1, wherein the reduced airflow velocity is at an intake point of the at least one airway.

32. The apparatus of claim 1, wherein, the reduced airflow velocity delivered through the at least one airway facilitates the volume of gas that passes through the at least one airway during inhalation of the patient to remain substantially the same or increase.

33-50. (canceled)

51. A patient interface comprising:
a mask configured to communicate with at least one airway of a patient, the mask including at least one aperture configured to permit entry of gas to the at least one airway of the patient;
an airflow member provided to the mask such that, in use, breathing by the patient reduces airflow and/or increases impedance during inhalation and/or expiration through the at least one airway; and
progressive airflow resistance structure to cooperate with the airflow member, such that, in use the flow of gas during inspiration and/or expiration is progressively decreased and/or impedance is progressively increased over a time period that includes multiple respiratory cycles of the patient.

52. The apparatus of claim 51, wherein the progressive airflow resistance structure comprises dissolvable structure including a water soluble polymer.

53. The apparatus of claim 52, wherein the water soluble polymer comprises corn starch.

54. The apparatus of claim 52, wherein the airflow member comprises indentations to increase the bonding strength between the dissolvable structure and the airflow member.

55-67. (canceled)

68. A respiratory assistance apparatus comprising:
an airflow member provided to the mask such that, in use, breathing by the patient reduces airflow and/or increases impedance during inhalation and/or expiration through the at least one airway; and
progressive airflow resistance structure to cooperate with the airflow member, such that, in use, the flow of gas during inspiration and/or expiration is progressively decreased and/or impedance is progressively increased over multiple respiratory cycles of the patient.

69-70. (canceled)

71. The apparatus of claim 1, wherein the airflow member, in use, is progressively adjusted over multiple respiratory cycles of the patient such that air intake through the airflow member during inhalation over the multiple respiratory cycles is decreased.

72. The apparatus of claim 71, wherein the multiple respiratory cycles are over a time period of between about 5 minutes and 1 hour.

73. The apparatus of claim 72, wherein the multiple respiratory cycles are over a time period that is between when the mask, in use, is secured to the patient and when the patient falls asleep.

74-77. (canceled)
